(19) United States
(12) Patent Application Publication
(10) Pub. No.: US 2022/0390450 A1
(43) Pub. Date: Dec. 8, 2022

(54) METHOD OF DETECTING OR MONITORING MINIMAL RESIDUAL DISEASE IN A MONOCLONAL GAMMOPATHY PATIENT

(71) Applicant: The Binding Site Group Limited, Birmingham (GB)

(72) Inventors: Gregg WALLIS, Birmingham (GB); Stephen HARDING, Birmingham (GB); Richard HUGHES, Birmingham (GB)

(21) Appl. No.: 17/774,086

(22) PCT Filed: Nov. 3, 2020
(86) PCT No.: PCT/GB2020/052785

§ 371 Date: May 3, 2022

(30) Foreign Application Priority Data
Nov. 4, 2019 (GB) 1916030.8

(51) Int. Cl. G01N 33/574; G01N 33/68

(52) U.S. Cl. CPC G01N 33/57426; G01N 33/6848; G01N 33/6857

(57) ABSTRACT

Method of Detecting or Monitoring Minimal Residual Disease The application describes a method of identifying minimal residual disease (MRD) in a monoclonal gammopathy patient, comprising detecting the presence or absence of a monoclonal free light chain (FLC) in a sample from the patient by mass spectrometry (MS).

The invention relates to a method of detecting or monitoring for the presence or absence of minimal residual disease (MRD) in a subject using mass spectrometry.

Antibody molecules (also known as immunoglobulins) have a twofold symmetry and typically are composed of two identical heavy chains and two identical light chains, each containing variable and constant domains. The variable domains of the heavy and light chains combine to form an antigen-binding site, so that both chains contribute to the antigen-binding specificity of the antibody molecule. The basic tetrameric structure of antibodies comprises two heavy chains covalently linked by a disulphide bond. Each heavy chain is in turn attached to a light chain, again via a disulphide bond. This produces a substantially “Y”-shaped molecule.

Heavy chains are the larger of the two types of chain found in antibodies, with typical molecular mass of 50,000-77,000 Da, compared with the smaller light chain (25,000 Da).

There are five main classes of heavy chain which are g, a, m, d and e which are the constituent heavy chains for: IgG, IgA, IgM, IgD and IgE respectively. IgG is the major immunoglobulin of normal human serum, accounting for 70-75% of the total immunoglobulin pool. This is the major antibody of secondary immune responses. It forms a single tetramer of two heavy chains plus two light chains.

IgM accounts for approximately 10% of the immunoglobulin pool. The molecules, together with J-chains, form a pentamer of five of the basic 4-chain structures. The individual heavy chains have a molecular weight of approximately 65,000 Da and the whole molecule has a molecular weight of about 970,000 Da. IgM is largely confined to the intravascular pool and is the predominant early antibody.

IgA represents 15-20% of human serum immunoglobulin pool. More than 80% of IgA occurs as a monomer. However, some of the IgA (secretory IgA) exists as a dimeric form.

IgD accounts for less than 1% of the total plasma immunoglobulin. IgD is found on the surface membrane of maturing B-cells.

IgE, although scarce in normal serum, is found on the surface membrane of basophils and mast-cells. It is associated with allergic diseases such as asthma and hay-fever.

In addition to the five main class or classes, there are four subclasses for IgG (IgG1, IgG2, IgG3 and IgG4). Additionally, there are two subclasses for IgA (IgA1 and IgA2).

There are two types of light chain: Lambda (λ) and Kappa (κ). There are approximately twice as many κ as λ molecules produced in humans, but this is quite different in some mammals. Each chain contains approximately 220 amino acids in a single polypeptide chain that is folded into one constant and one variable domain. Plasma cells produce one of the five heavy chain types together with either κ or λ molecules. There is normally approximately 40% excess free light chain production over heavy chain synthesis. Where the light chain molecules are not bound to heavy chain molecules, they are known as “free light chain (FLC) molecules”. The κ light chains are usually found as monomers. The λ light chains tend to form dimers.

There are a number of proliferative diseases associated with antibody producing cells.

In many such proliferative diseases a plasma cell proliferates to form a monoclonal tumour of identical plasma cells. This results in production of large amounts of identical immunoglobulins and is known as a monoclonal gammopathy.

Diseases such as myeloma and primary systemic amyloidosis (AL amyloidosis) account for approximately 1.5% and 0.3% respectively of cancer deaths in the United Kingdom. Multiple myeloma is the second-most common form of haematological malignancy after non-Hodgkin lymphoma. In Caucasian populations the incidence is approximately 40 per million per year. Conventionally, the diagnosis of multiple myeloma is based on the presence of excess monoclonal plasma cells in the bone marrow, monoclonal immunoglobulins in the serum or urine and related organ or tissue impairment such as hypercalcaemia, renal insufficiency, anaemia or bone lesions. Normal plasma cell content of the bone marrow is about 1%, while in multiple myeloma the content is typically greater than 10%, frequently greater than 30%, but may be over 90%.

AL amyloidosis is a protein conformation disorder characterised by the accumulation of monoclonal free light chain fragments as amyloid deposits. Typically, these patients present with heart or renal failure but peripheral nerves and other organs may also be involved.

There are a number of other diseases which can be identified by the presence of monoclonal immunoglobulins within the blood stream, or indeed urine, of a patient. These include plasmacytoma and extramedullary plasmacytoma, a plasma cell tumour that arises outside the bone marrow and can occur in any organ. When present, the monoclonal protein is typically IgA. Multiple solitary plasmacytomas may occur with or without evidence of multiple myeloma. Waldenström's macroglobulinaemia is a low-grade lymphoproliferative disorder that is associated with the production of monoclonal IgM. There are approximately 1,500 new cases per year in the USA and 300 in the UK. Serum IgM quantification is important for both diagnosis and monitoring. B-cell non-Hodgkin lymphomas cause approximately 2.6% of all cancer deaths in the UK and monoclonal immunoglobulins have been identified in the serum of about 10-15% of patients using standard electrophoresis methods. Initial reports indicate that monoclonal free light chains can be detected in the urine of 60-70% of patients. In B-cell chronic lymphocytic leukaemia monoclonal proteins have been identified by free light chain immunoassay.

Additionally, there are so-called MGUS conditions. These are monoclonal gammopathy of undetermined significance. This term denotes the unexpected presence of a monoclonal intact immunoglobulin in individuals who have no evidence of multiple myeloma, AL amyloidosis, Waldenström's macroglobulinaemia, etc. MGUS may be found in 1% of the population over 50 years, 3% over 70 years and up to 10% over 80 years of age. Most of these are IgG- or IgM-related, although more rarely IgA-related or bi-clonal. Although most people with MGUS die from unrelated diseases, MGUS may transform into malignant monoclonal gammopathies.

In at least some cases for the diseases highlighted above, the diseases present abnormal concentrations of monoclonal immunoglobulins or free light chains. Where a disease produces the abnormal replication of a plasma cell, this often results in the production of more immunoglobulins by that type of cell as that “monoclone” multiplies and appears in the blood.

Immunoassays rely on the interaction between a protein (antigen) and an antibody, or fragment, specific to that protein. Conventionally immunoassays are used to detect the amount of total LC (that is bound plus non-heavy chain bound LC) or free light chain (FLC) not bound to heavy chains, in a sample.

Serum monoclonal proteins may be detected and quantified by serum protein electrophoresis (SPE), with the concentration of each type of protein measured by scanning densitometry of the SPE gel. Similarly urine proteins may be measured by urine protein electrophoresis (UPE), following a pre-analytical concentration step capillary zone electrophoresis (CZE) provides an alternative method of quantifying serum proteins. Limitations of SPE, UPE and CZE include the incidence of so-called “false-negatives” and limited sensitivity. ELISA may also be used but is also known to underestimate polymeric light chains.

Nephelometric and turbidimetric assays, for example using latex-enhanced methods are also used to detect and quantitate the amount of heavy chain containing immunoglobulins, total LC and FLC in samples.

Heavy chains may be quantitated using anti-heavy chain class specific antibodies, such as anti-IgG, anti-IgA or anti-IgM. Alternatively, anti-heavy chain class light chain type specific antibodies, specific for, for example IgAκ or IgAλ, such as those produced under the trademark Hevylite™ (The Binding Site, Birmingham, United Kingdom) have been used.

Total light chains, such as the total amount of kappa or lambda light chains or the total amount of kappa plus lambda light chains detect the amount of both light chains bound to heavy chains, and those not bound to light chains (such as serum free light chains sFLCs). For example, total kappa detects IgGκ+IgAκ+IgMκ+IgDκ+IgEκ+κFLC. Total κ and total λ assays are often too insensitive to detect monoclonal immunoglobulin or FLC unless there are gross amounts of monoclonal protein present. This is due to high background concentrations of polyclonal bound light chains which interfere with such assays.

FLCs, such as sFLCs may be measured using free light chain specific antibodies such as anti-free kappa or anti-free lambda specific antibodies. These include antibodies produced under the tradename Freelite™ by The Binding Site, Birmingham, United Kingdom. Such assays are particularly of interest as they have sensitivities as low as 0.25-3 mg/L for FLC compared to 0.5 g/L for SPE and 0.25 g/L for CZE.

Mass spectrometry (MS) allows the separation of analytes by mass/charge (m/z). Polyclonal light chains have a varied set of masses so typically produce a normally-distributed bell-shaped curve of m/z against peak intensity. Monoclonal light chains resolve as a sharp peak on the bell curve. One example of the use of MS to detect free light chain is WO 2018/215768.

Mass spectrometry has a number of advantages for the detection of immunoglobulins compared to serum plasma electrophoresis (SPE) and immunofixation electrophoresis (IFE). SPE has a limit of detection of approximately 1000 mg/L and limit of quantification of 1000-3000 mg/L. IFE has a limit of detection of 50-100 mg/L and is not quantifiable. In contrast MS can detect 1 mg/L of IgA, IgM or IgA and quantitate 24 mg/L of IgG or 8 mg/L of IgA or IgM. MS can also detect and quantitate low concentrations of free light chains, for example with an average sensitivity of 2 mg/L for a FLC clone in a normal polyclonal background.

This allows MS to detect monoclonal light chains below the normal range of concentration of the polyclonal light chains seen in patients. The monoclonal light chains being seen as a sharply resolved peak in the spread of polyclonal light chains of different masses. This makes the monoclonal light chain readily detectable against the polyclonal background light chains.

One problem associated with the treatment of monoclonal gammopathies such as multiple myeloma, is that sometimes a small number of malignant cells are left after front line therapy. Minimal residual disease (MRD) refers to presence of a small number of malignant cells below the limit of detection available with conventional morphological assessment. In multiple myeloma, MRD refers to myeloma cells present, usually in the bone marrow, after clinical response has been measured and the patient is in remission. These residual cells are clinically relevant because they may lead to disease progression and relapse. It is therefore important to monitor a subject for MRD. Residual malignant cells may be present in the central nervous system, for example in lymphocytic leukemia. The earlier a subject has MRD identified, the sooner the subject is able to be treated and the better the long term prognosis of the subject. Such testing conventionally comprises sampling bone marrow and testing bone marrow aspirates, for example by staining of plasma cells or the detection of VDJ heavy chains in the bone marrow aspirate by ASO-PCR or NGS. This is an invasive technique requiring an often-painful procedure to be carried out on the subject, which limits the frequency of occurrence during patient monitoring

Patients may be monitored for relapse, once they are in remission and have been shown to be negative for MRD. The frequency and sensitivity of monitoring is dependent on the technique used, for example frequent invasive BM sampling will be avoided.

The Applicant realized that there is a need to produce an improved, sensitive, method of monitoring for MRD which reduces the use of invasive procedures.

The Applicant realized that monitoring of immunoglobulins after treatment is complicated by the half-life of different immunoglobulins in the blood:

| Immunoglobulin | Half-Life | |
|---|---|---|
| IgG | 21 | days |
| IgA | 6 | days |
| IgM | 5 | days |
| IgD | 2-8 | days |
| IgE | 1-5 | days |
| Free Light Chain | 2-6 | hours |

This means that, for example, for 1000 mg/L of each type of immunoglobulin approximately 900 mg/L if IgG will remain after 5 days, 550 mg/L IgA, 150 mg/L IgD, but no FLC.

Selecting FLC as a way of monitoring the effectiveness of the treatment of a subject is better than other types of immunoglobulins. It means that monoclonal FLC is observed against a background of polyclonal FLC with a short half-life, and that is a better representation of the current disease status of the subject due to the short half-life of FLC. Monitoring heavy chains, such as IgG means that whilst a clone may have been successfully treated, residual IgG will remain in the blood stream for many days and not give a true representation of the status of a patient.

The sensitivity of MS allows the detection of FLC from MRD to be detected, without the need of invasive sampling of a subject's bone marrow.

The evolution of myeloma testing in clinical chemistry with mass spectrometry is reviewed by Murray D. L. and Willrich M. A. V., JALM, 2019, 474-476. This focused on the isolation and detection of monoclonal proteins by immunoprecipitation with anti-IgG, anti-IgA, anti-IgM, anti-total kappa and anti-total lambda antibodies (G, A, M, K, L monitoring). The latter two antibodies detect kappa or lambda light chains both as bound to heavy chain plus free, dissociated, light chains, so includes, light chains from immunoglobulins with long half-lives.

Accordingly, the invention provides a method of identifying minimal residual disease (MRD) in a monoclonal gammopathy patient, comprising detecting the presence or absence of a monoclonal free light chain (FLC) in a sample from the patient by mass spectrometry (MS). Typically, the subject has had the monoclonal gammopathy treated prior to the screening. The subject may have been in remission prior to detecting the MRD.

The subject may be treated for a number of cycles of chemotherapy 1, 2, 3 . . . up to 6, 7 or 8 cycles. Following this the testing may continue once the treatment cycles have been completed

The treatment may, for example, include treatment with bortezomib (Velcade), carfilzomib (Kyprolis) or ixazomib (Nilaro) or other monoclonal antibodies. Alternatively, the drug may be, for example, thalidomide, lenalidomide (Revlimid) or pomalidomide (Pomalyst). Chemotherapy drugs may also be used, for example, to kill fast growing cells such as myeloma cells. Corticosteroids, such as prednisone and dexamethasone, may also be used. Moreover, bone marrow transplants may also be used to replace the diseased bone marrow with healthy bone marrow.

If no FLC clone is detected by the first screening, then the subject may be monitored by G, A, M, K. L mass spectrometry monitoring, such as that discussed above.

If a FLC clone is detected, for example by the presence or absence of the monoclonal FLC peak against the background polyclonal free light chains, then the clone may be monitored by screening a further sample for FLC by MS after a time interval. The time interval may be for example between 3 days and 4 months, more typically, 14 days-3 months. The sample may be of a subject in remission who is being monitored for MRD, for example, many months after treatment. If an FLC clone is detected in that further sample then the clone is monitored by screening at least one still further sample for FLC by MS after a further time interval, such as the time interval discussed above.

Preferably both the FLC kappa and FLC lambda should be assessed by MS. Where the light chain type of the FLC clone is known, at least the relevant FLC type should be assessed by MS. For example a kappa FLC clone should be assessed by MS for monoclonal Kappa FLCs and a lambda FLC clone should be assessed by MS for monoclonal lambda FLCs, preferably the patient should be assessed for both kappa and lambda FLCs.

Where no clone is detected in such further samples, then (a) either a further sample is tested after a time interval (such as time interval as discussed above) for FLC by MS to see if a clone is detected or (b) a sample of bone marrow is tested for a clone. The subject may be further tested one or more times at intervals of time, as discussed above, until no clone is detected by screening for FLC by MS. Once no clone is detected, then a sample of bone marrow may be tested for a clone.

Where a patient has been shown to be in remission and have been shown to be negative for MRD, they may be monitored for clonal FLC by mass spec to identify the occurrence or absence of relapse to MRD.

The method of detection of FLC by MS is generally known in the art. The mass spectrometry method used may be liquid chromatography MS (LC-MS) or MALDI-TOF.

A sample may be a biological sample, such as a sample of blood, serum, plasma, cerebrospinal fluid or urine, most typically blood, serum or plasma. The monoclonal gammopathy may be selected from multiple myeloma, AL amyloidosis, plasmacytoma, Waldenström's macroglobulinaemia, B-cell non-Hodgkin lymphoma and B-cell chronic lymphocytic leukaemia, such as acute lymphocytic leukaemia.

A subject may be monitored by one or more additional techniques prior to monitoring immunoglobulins in the sample by detecting FLC by MS. Such methods may include nephelometry, turbidimetry and ELISA, wherein the FLC-MS assay is used once the FLC is observed to return to a predetermined normal level in the subject.

The immunoglobulins in the subject may also be monitored prior to detecting FLC by MS using serum plasma electrophoresis or immunofixation electrophoresis, where the FLC-MS screening is carried out if the electrophoresis result is identified not to be within the normal range for a healthy subject.

The invention will now be described by way of example only with reference to the following examples:

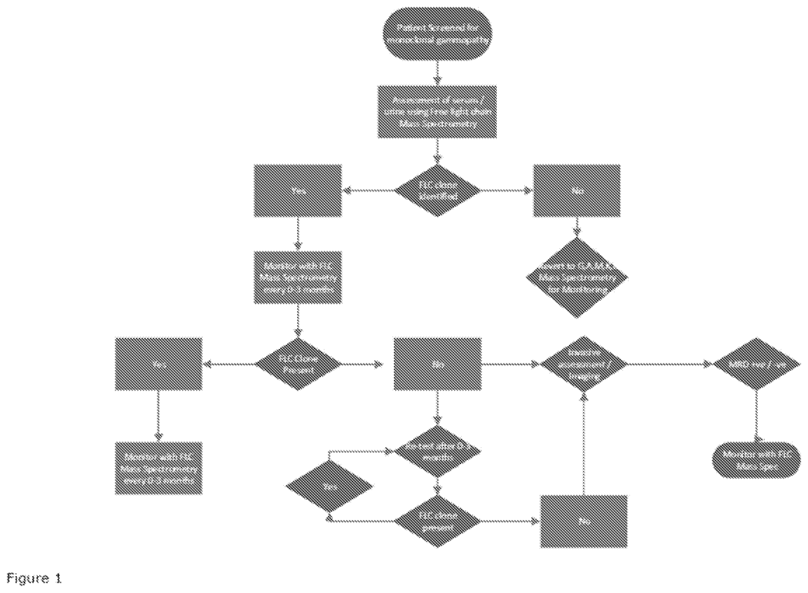

FIG. 1 shows a flow diagram showing the typical steps in determining which assay to use to detect MRD.

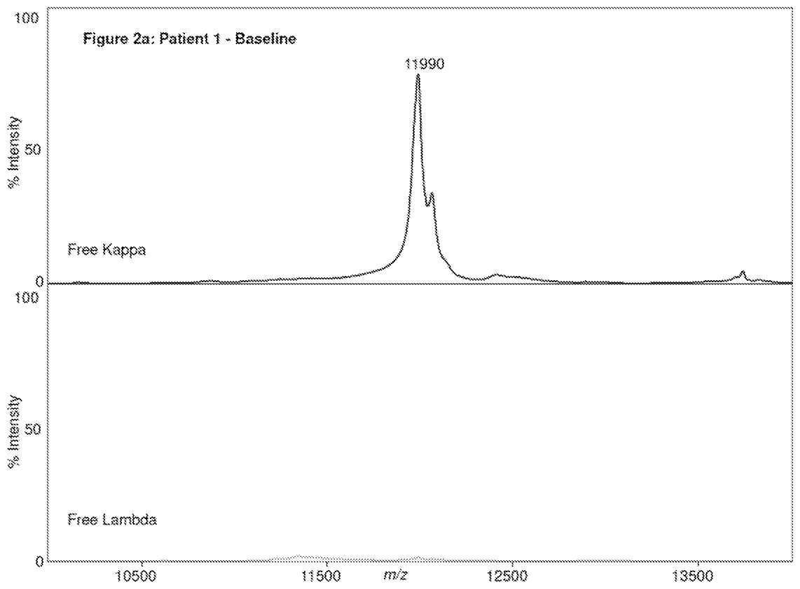

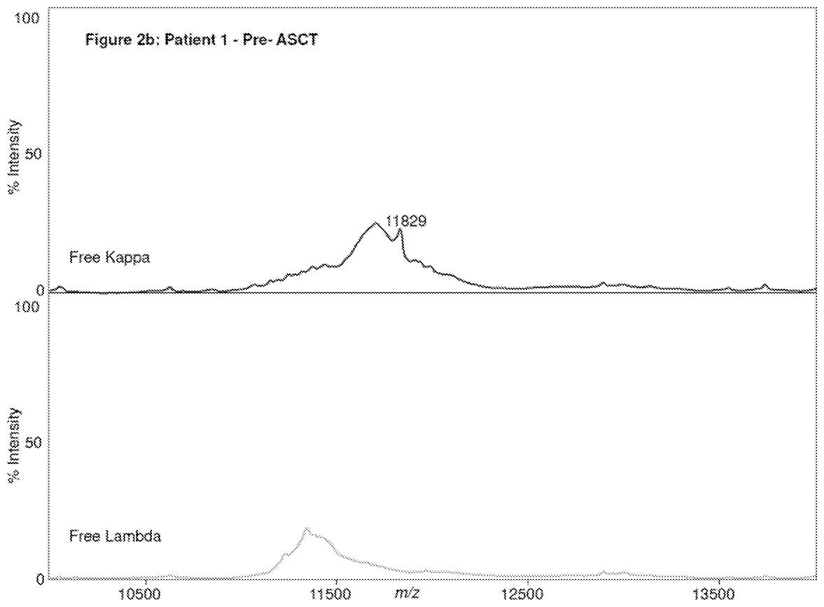

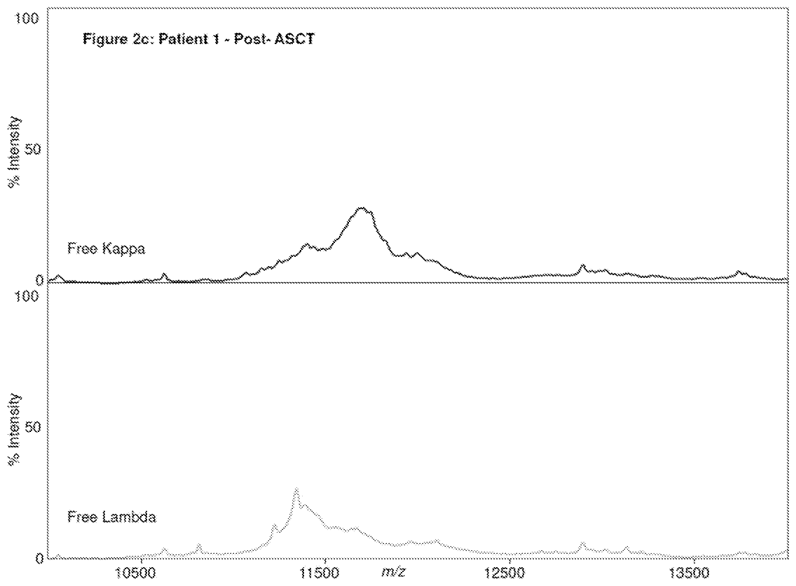

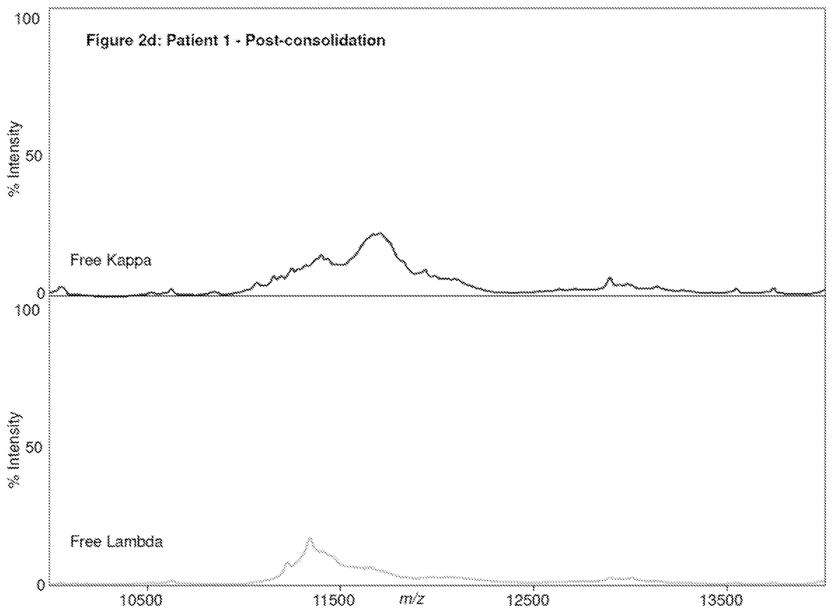

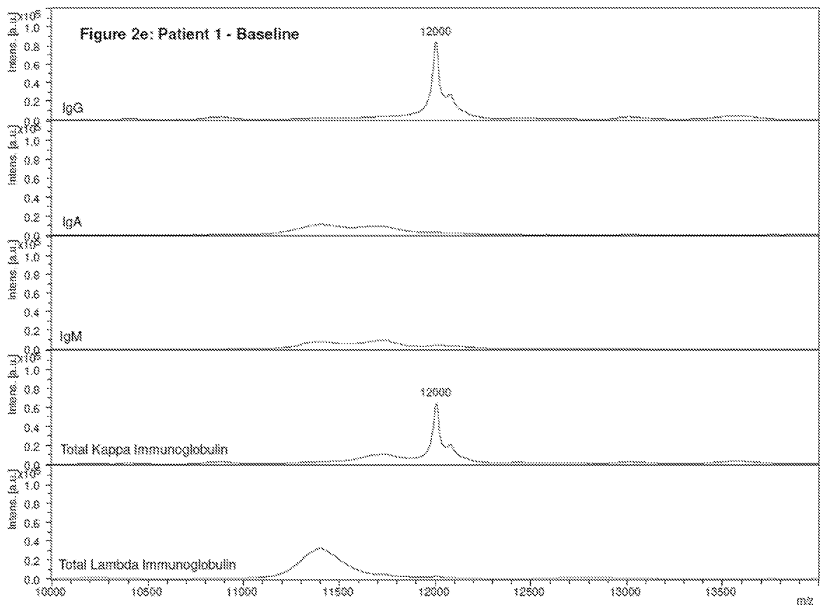

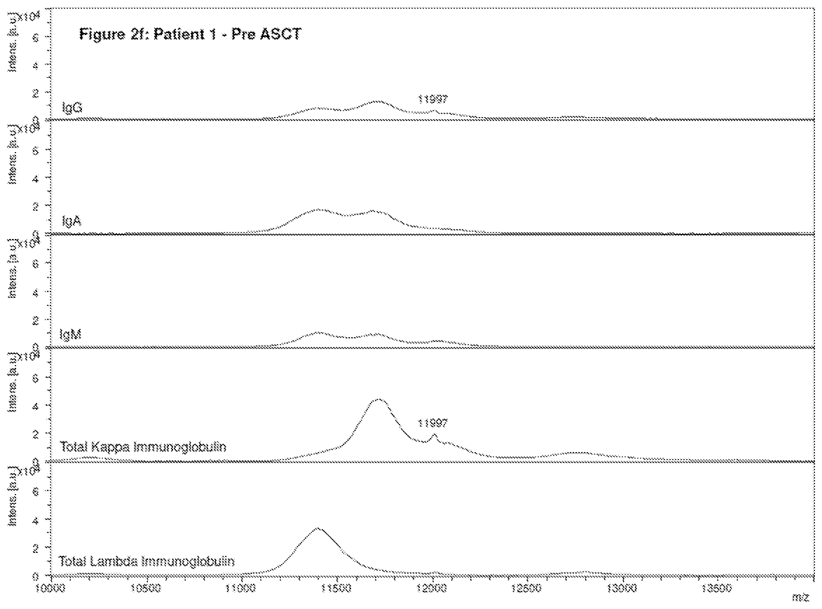

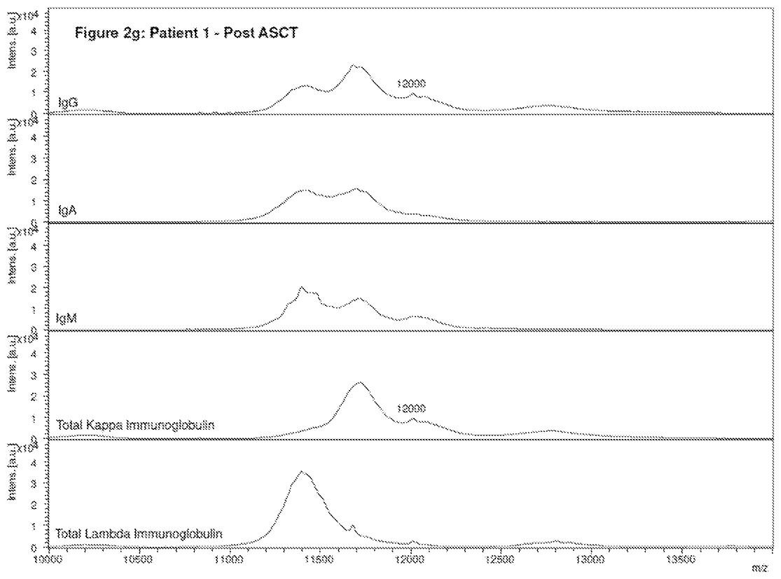

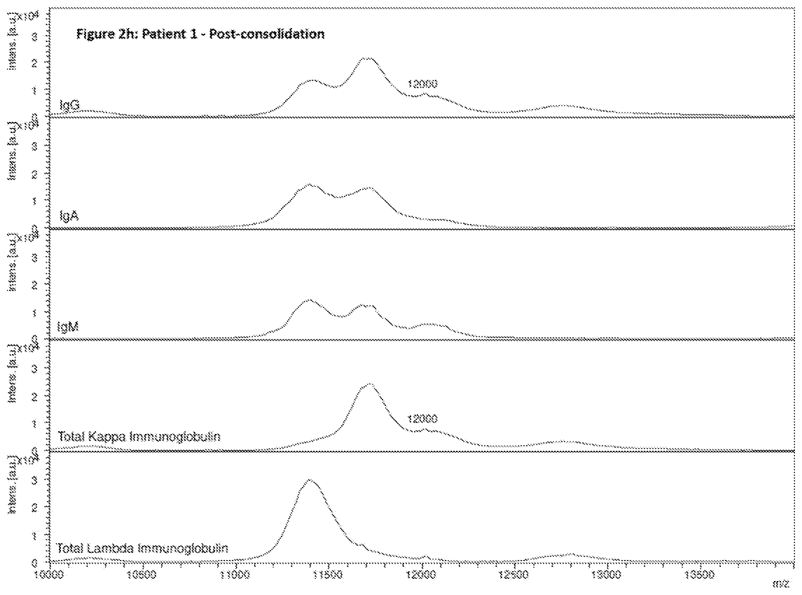

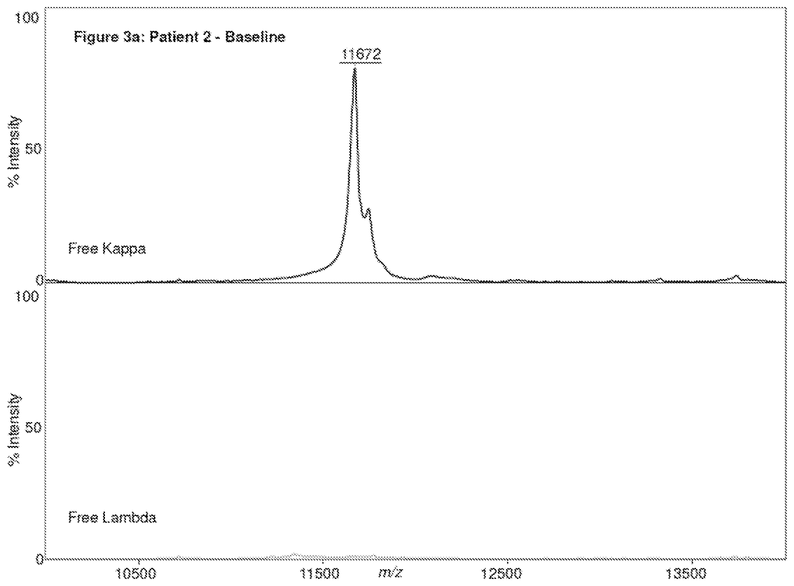

FIG. 2 shows a patient (1) with a free Kappa monoclonal protein peak of m/z 11990 at presentation or baseline (a); reduced pre-ASCT (Autologous Stem Cell Transplant) (b); post-ASCT (c) and post-consolidation (d); FIG. 2 (e-h) shows QIP-MS of IgG, IgA, IgM, total kappa and total lambda at baseline (e), pre-ACST (f), post-ACST (g) and post-consolidation (h).

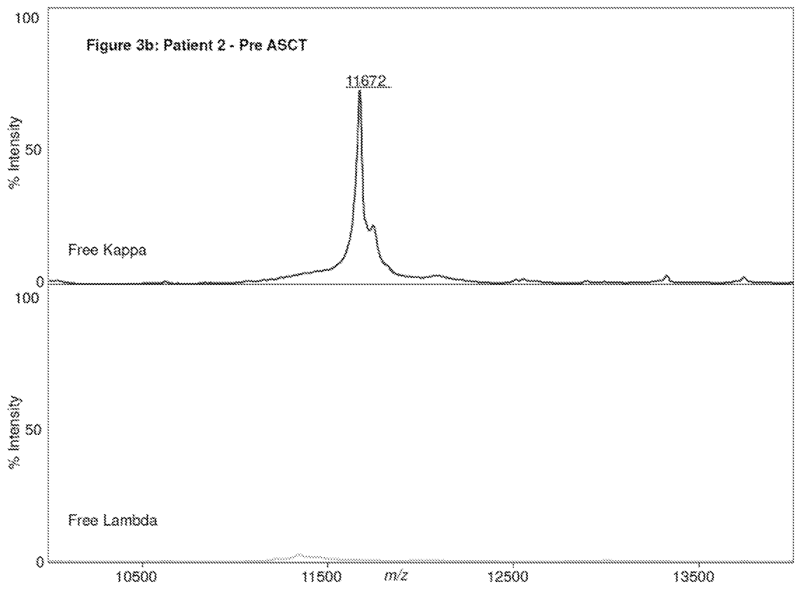

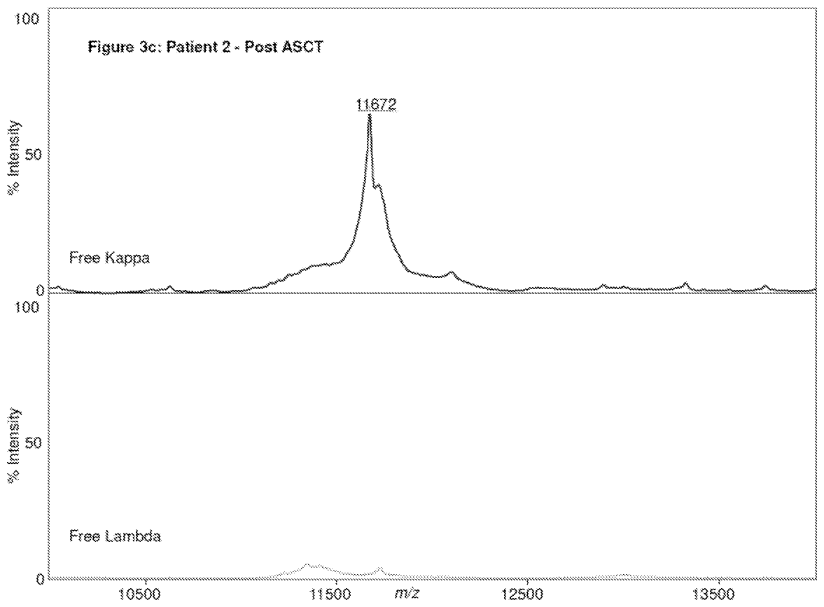

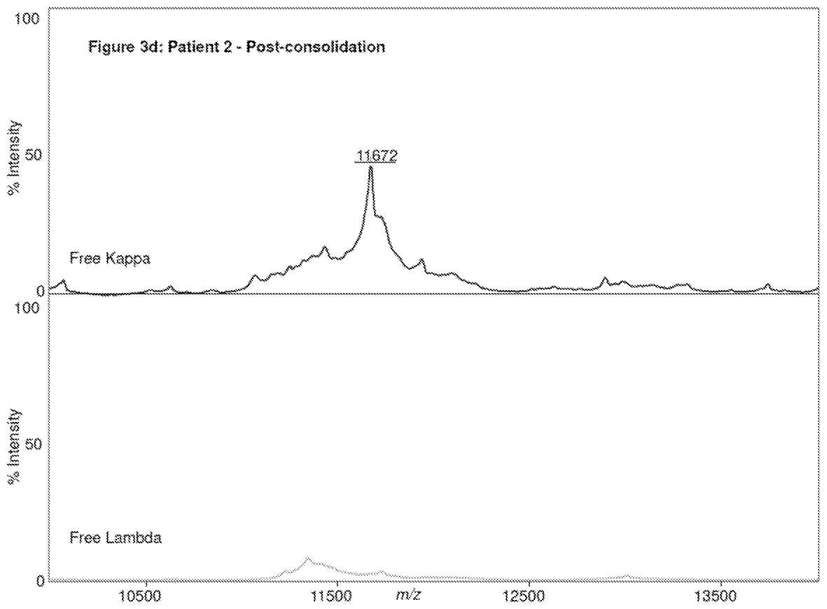

FIG. 3 shows a patient (2) with a free Kappa monoclonal protein peak of m/z 11672 at presentation or baseline (a); reduced pre-ASCT (Autologous Stem Cell Transplant) (b); post-ASCT (c) and post consolidation (d).

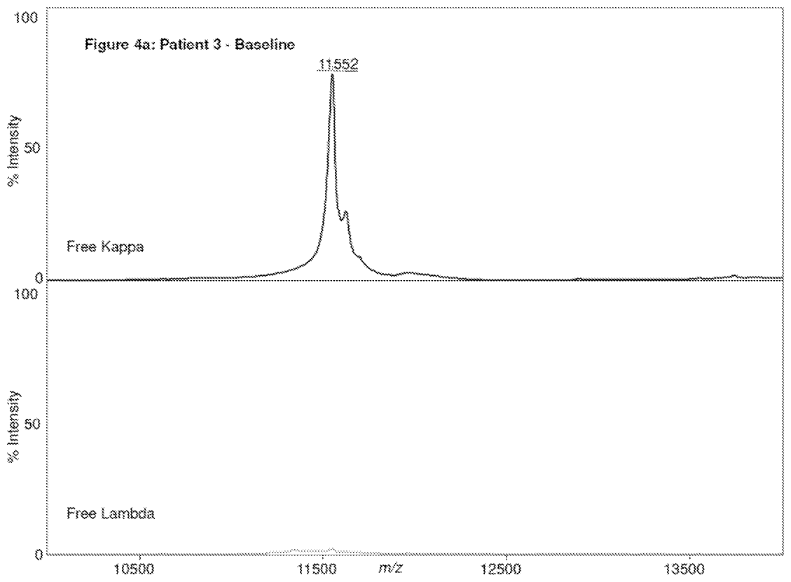

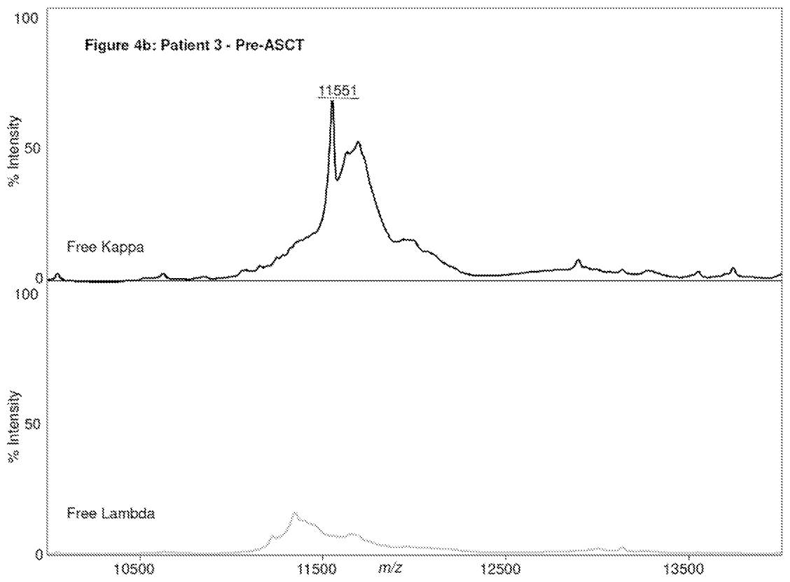

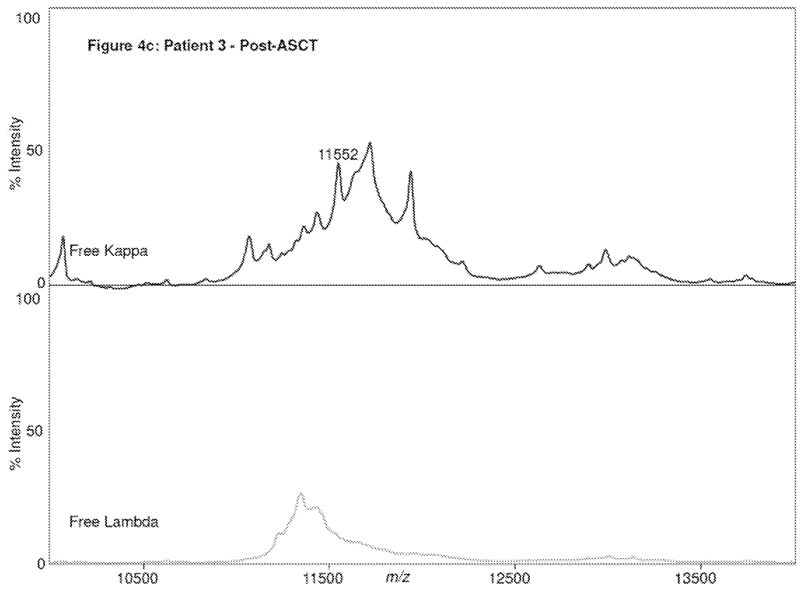

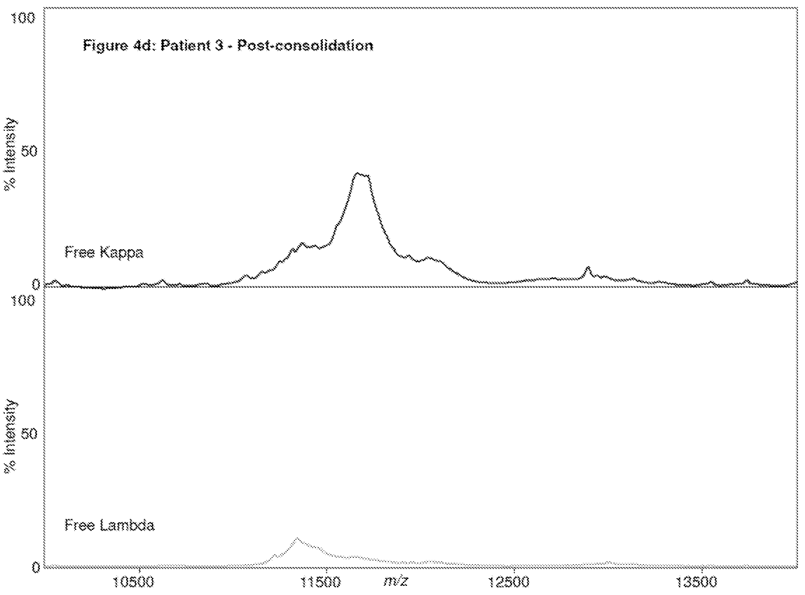

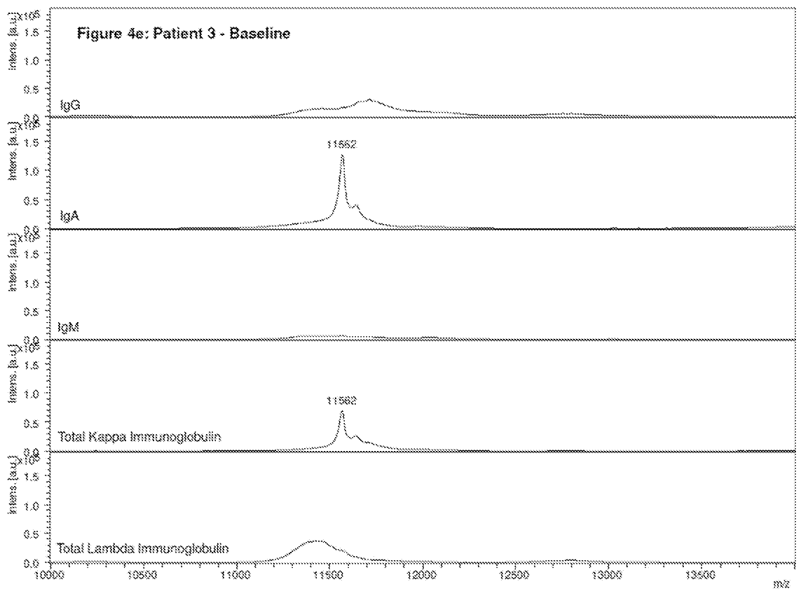

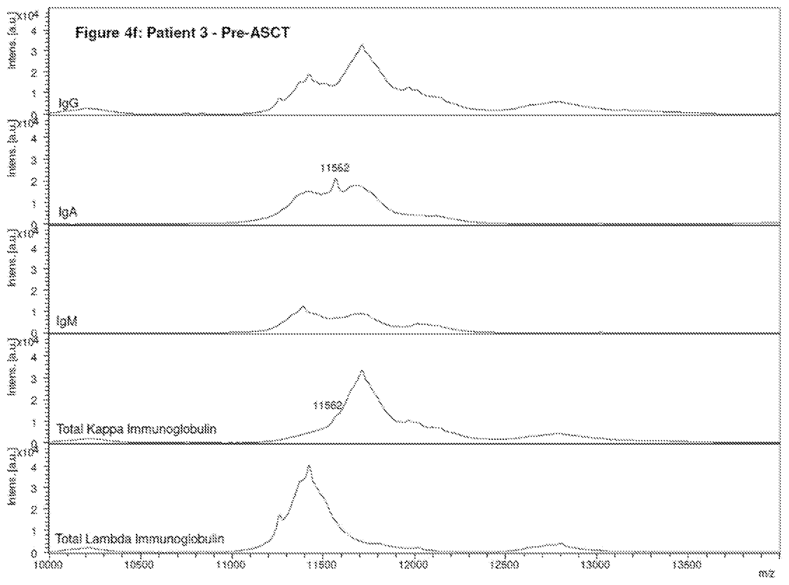

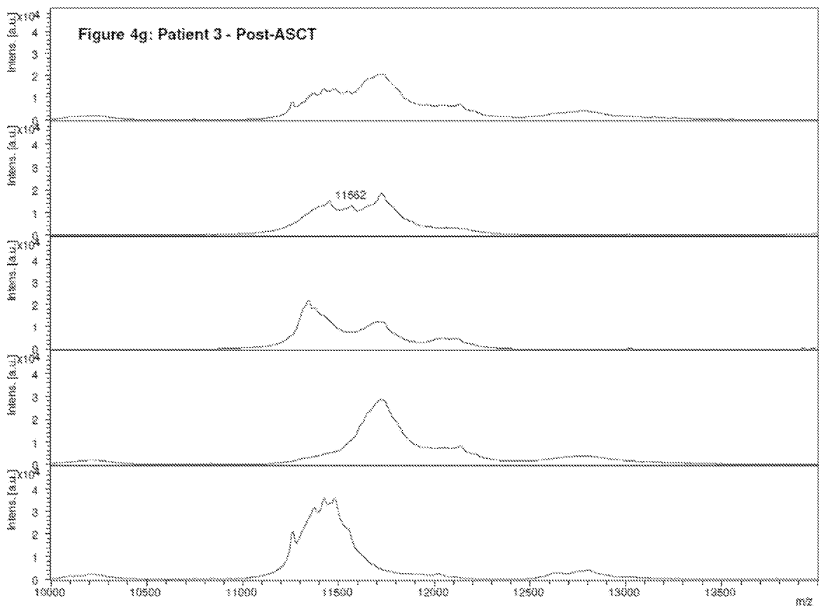

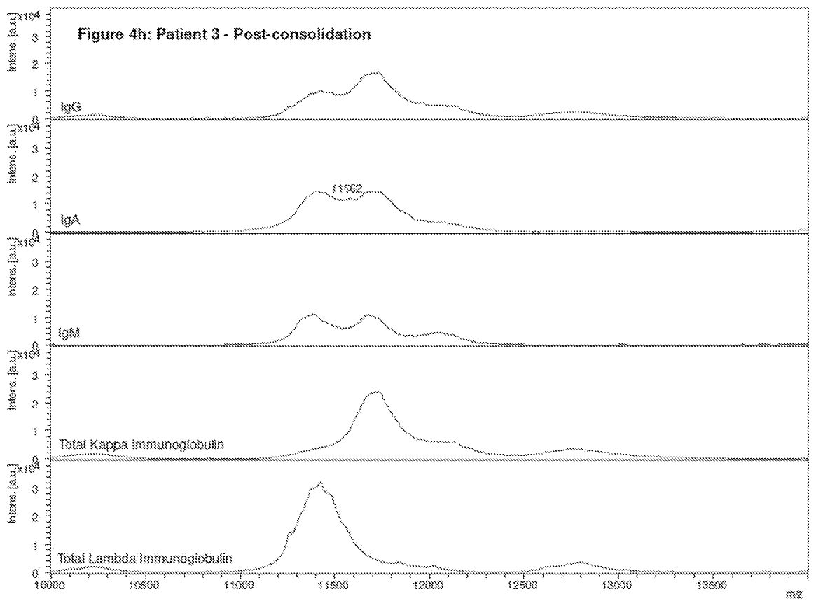

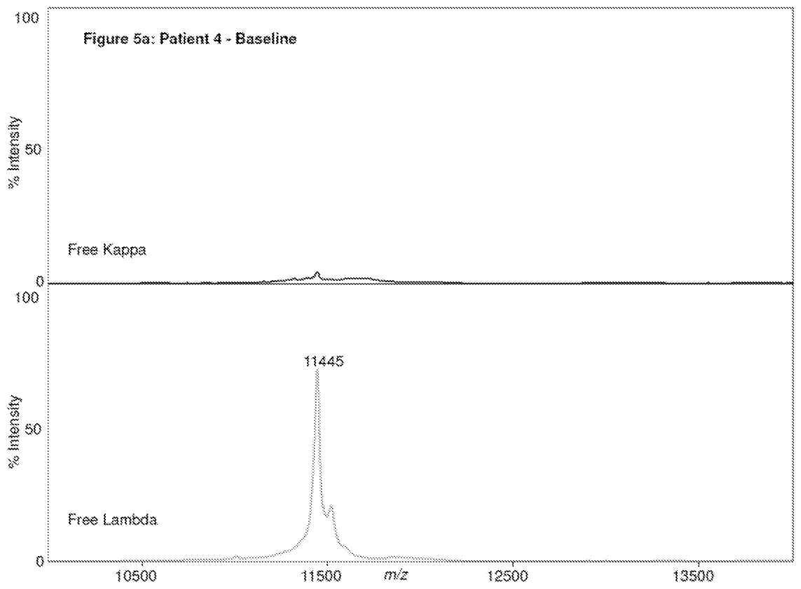

FIG. 4 shows a patient (3) with a free Kappa monoclonal protein peak of m/z 11552 at presentation or baseline (a); reduced pre-ASCT (Autologous Stem Cell Transplant) (b); post-ASCT (c); post-consolidation (d); FIG. 4 (d-f) shows QIP-MS of IgG, IgA, IgM, total kappa and total lambda at baseline (e), pre-ASCT (f), post-ACST (g), post consolidation(h).

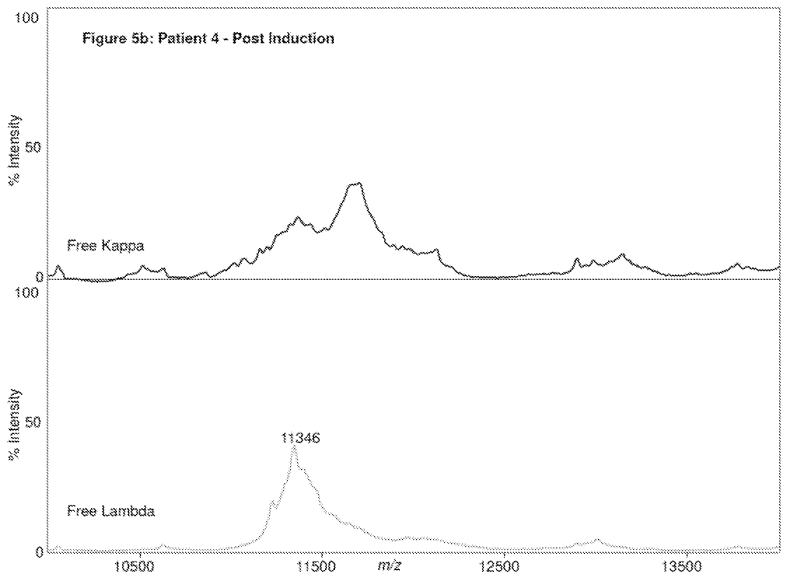

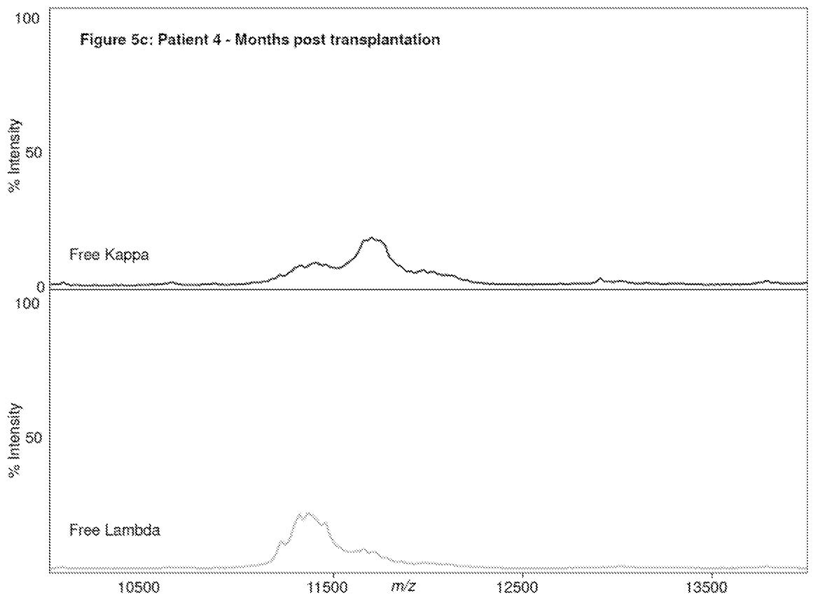

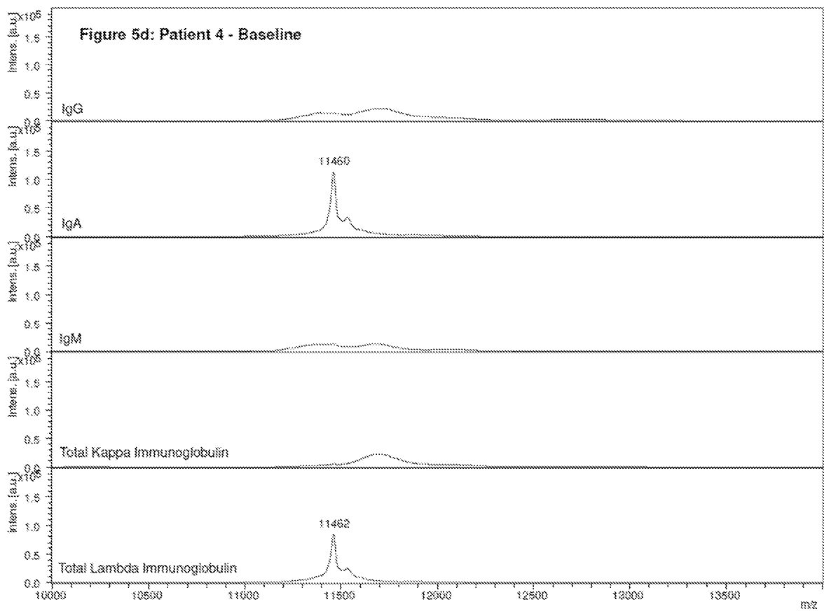

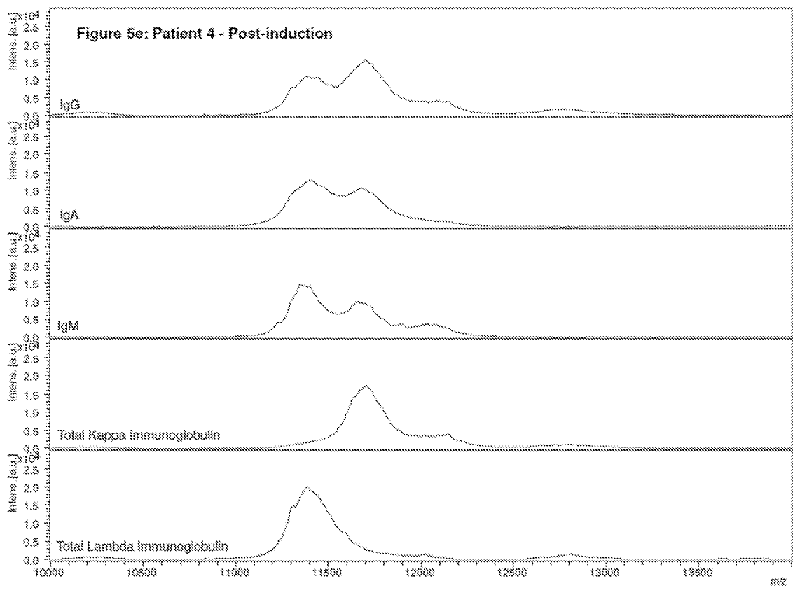

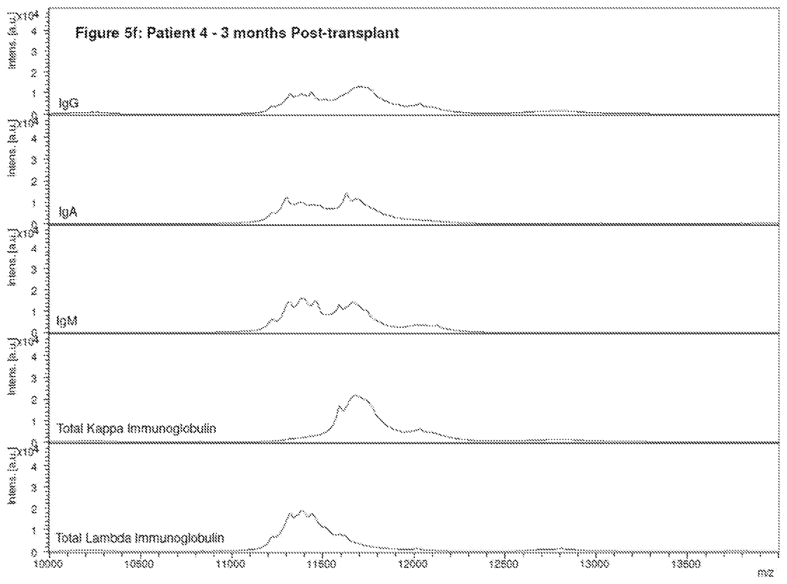

FIG. 5 shows a patient (4) with a free lambda monoclonal peak of m/z 11445 at baseline, post-induction (b), 3 months post transplantation (c), FIG. 5 (d-f) shows QIP-MS of IgG, IgA, IgM, total kappa and total lambda at baseline (d), post induction (e), 3 months post-transplantation (f).

Typically the patient has been previously diagnosed with a monoclonal gammopathy such as multiple myeloma. The patient is then typically treated by one or more techniques known in the art, as discussed above. The patient may be monitored by nephelometry or turbidimetry or ELISA, to detect free light chains in the subject. Such free light assays include the assay sold under the tradename Freelite™ (The Binding Site Group Limited, Birmingham, England). The latter technique uses latex-enhanced nephelometry with anti-FLC antibodies. Once the amount of FLC is observed to return to a normal level for a normal healthy subject, then the blood, serum, plasma or urine from a patient may be screened for free light chains by mass spectrometry. Alternatively, SPEP/IFE may be used to also monitor FLC for other serum proteins. If the serum proteins appear to be abnormal, then FLCs may be detected by mass spectrometry, such as described in WO 2018/215768, incorporated herein in its entirety.

Once the FLC has been screened by mass spectrometry an FLC clone may be detected as a peak on the shoulder of the curve of different masses of polyclonal light chains in the background within the sample from the subject. If such a clone is identified, then this is typically monitored for FLC mass spectrometry, for example, every 0-3 months to see if the FLC clone is again present. If it is then further monitoring for FLC by mass spectrometry may be used. If no FLC clone is present after the first round of screening by mass spectrometry, then typically the subject is monitored by more conventional G, A, M, K, L mass spectrometry, such as reviewed by Murray and Willrich (Supra).

Where a further screen has taken place, then if no clone is present then alternatively further testing by mass spectrometry may occur after a period of typically 0-4, or 0-3 months. If an FLC clone is again present then retesting is then carried out. Alternatively, if no clone is present before or after that additional retesting, then a sample of bone marrow may be removed and tested as discussed above.

After the test, if MRD is detected then one or more further rounds of monitoring for FLC by mass spectrometry may occur.

Where a patient has been shown to be in remission and have been shown to be negative for MRD, they will be monitored for clonal FLC by mass spec to identify the occurrence or absence of relapse to MRD. Where a FLC clone is shown to be present by MS then this may act as a trigger for the treating clinician to carry out additional assessments.

### EXAMPLES

### Methods

The standard of care for multiple myeloma patients is to receive high-dose chemotherapy with autologous stem cell rescue—or autologous stem cell transplant (ASCT)—after completion of induction therapy. ASCT can provide significant reduction in disease, extending patient survival. Many patients will continue to receive maintenance or post-consolidation therapy post transplantation to reduce the risk of relapse. To demonstrate the detection and sensitivity of monoclonal paraproteins, throughout these phases using the mass spectrometry technology referred to in the application, longitudinal clinical samples were obtained from 4 patients undergoing diagnosis and treatment for Multiple Myeloma. Each sample was diluted in phosphate buffered saline+tween (PBST) to appropriate levels and immunocaptured using 7 different magnetic bead types; Free Kappa specificity and Free Lambda specificity or, total immunoglobulin (IgG specificity, IgA specificity, IgM specificity, Total Kappa specificity, Total Lambda specificity). The captured samples were washed sequentially (PBST and water) and then eluted using 5% acetic Acid and 20 mM tris(2-carboxyethyl) phosphine (TCEP). The eluted samples were co-spotted onto MALDI target plates alongside matrix (α-Cyano-4-hydroxycinnamic acid in acetonitrile and water spiked with trifluoroacetic acid) and dried to allow for MALDI analysis. The resulted mass spectra were acquired on a Bruker Microflex Biotyper MALDI Mass Spectrometer over the mass-range 5000-32000 m/z in the positive ion mode. The data is presented for the +2charge state ions of immunoglobulin light chains.

### Results

Patient 1 (FIGS. 2a-2h). The presentation or baseline sample's mass spectra showed a clear monoclonal protein peak of m/z 11990 in the Free-kappa spectra. Indicating the presence of a Free kappa paraprotein. This peak reduced in intensity at the pre-ASCT stage and had disappeared post-ASCT and post-consolidation. No monoclonal peaks were seen in the Free-lambda spectra. Similarly, the same monoclonal peak was observed in the IgG and total kappa immunoglobulin spectra. Indicating the presence of an IgGK+free kappa paraprotein. This data shows that both free-light chain or total-light chain MALDI-TOF-MS can be employed successful to follow successful therapy over many months in this patient.

In this example the total immunoglobulin light chain assessment is seen to be more sensitive for the monoclonal protein, however, this observation may be due to the difference in clearance between the intact monoclonal protein and the Free light chains. Free light chains are relatively small molecules that are readily cleared by the kidney and so have a very short half-life (Kappa FLCs—2 hours; lambda FLCs 4-6 hours) and will therefore clear relatively quickly when there is no longer any production by an aberrant plasma cell clone. In contrast, Intact immunoglobulins are relatively large and are not readily cleared by the kidney, furthermore IgG immunoglobulins are recycled via the FcRN receptor and their half-life is very long (IgA and IgM 5-6 days; IgG ˜21 days).

Patient 2 (FIGS. 3a-3d). The baseline mass spectra showed a clear monoclonal peak of m/z 11672 in the Free-kappa spectra. Indicating the presence of a Free kappa paraprotein. This peak reduced in intensity in the pre-ASCT, post-ASCT and post-consolidation stage but did not entirely disappear. No monoclonal peaks were seen in the Free-lambda spectra. This shows that free-light chain in the absence of total-immunoglobulin MALDI-TOF-MS can be monitored by mass spectrometry to follow therapy over many months and years in a patient that has not completely responded to a therapy regime.

Patient 3 (FIGS. 4a-4h). The baseline mass spectra showed a clear monoclonal peak of m/z 11552 in the Free-kappa spectra. Indicating the presence of a Free kappa paraprotein. This peak diminished in intensity at the pre-ASCT stage and post-ASCT stage. The monoclonal FLC was not shown to be present in the sample from the post-consolidation stage of treatment. No monoclonal peaks were seen in the Free-lambda spectra. Correspondingly, the same monoclonal m/z peak was observed in the IgA and total Kappa spectra from baseline and pre-ASCT samples but had largely disappeared by the post-ASCT phase. This shows that both free-light chain or total-light chain MALDI-TOF-MS can be employed to follow successful therapy in this patient. The FLC assessment is shown to be more sensitive in this patient sample series and the assessment of FLCs avoids having to consider the impact of the longer half-life of intact immunoglobulins when using the result to interpret the sensitivity for the presence of an aberrant plasma cell producing a monoclonal protein.

Patient 4 (FIGS. 5a-5f). The baseline mass spectra showed a clear monoclonal peak of m/z 11445 in the Free-lambda spectra. Indicating the presence of a Free lambda paraprotein. This peak reduced in intensity at the post induction (therapy) stage and had virtually disappeared 3 months after transplant. No monoclonal peaks were seen in the Free-kappa spectra. The same monoclonal peak was observed in the IgA and total lambda baseline spectra, suggesting the presence of an IgAL and free lambda paraprotein, but this had largely disappeared by the post-induction phase. This indicates that both free-light lambda chain or total-light chain MALDI-TOF-MS can be employed successful to follow successful therapy in this lambda-patient. The FLC assessment is shown to be more sensitive in this patient sample series and the assessment of FLCs avoids having to consider the impact of the longer half-life of intact immunoglobulins when using the result to interpret the sensitivity for the presence of an aberrant plasma cell producing a monoclonal protein.

The data demonstrates the utility of the mass spectrometry method when monitoring for the presence or absence of an aberrant monoclonal protein producing plasma cell clone during longitudinal therapy. It indicates the additional sensitivity of FLCs over assessment of intact immunoglobulins. And demonstrates this is a useful approach to employ prior to applying more sensitive and potentially more invasive methods to assess for MRD.

1. A method of identifying minimal residual disease (MRD) in a monoclonal gammopathy patient, comprising detecting the presence or absence of a monoclonal free light chain (FLC clone) in a sample from the patient by mass spectrometry (MS).

2. A method according to claim 1, wherein the subject has had the monoclonal gammopathy treated prior to the screening and/or has been in remission from the monoclonal gammopathy.

3. A method according to claim 1, wherein if no FLC clone is detected then the subject is then monitored by detecting G, A, M, K, by MS.

4. A method according to claim 1, wherein if a FLC clone is detected then the clone is monitored by screening a further sample for a FLC clone by MS after a time interval.

5. A method according to claim 1, wherein if an FLC clone is detected then the clone is monitored by screening a still further sample for a FLC clone by MS after a time interval.

6. A method according to claim 1, wherein if no clone is detected then (a) either a further sample is tested after a time interval for a FLC clone by MS to see if a clone is detected or (b) a sample of bone marrow is tested for a FLC clone.

7. A method according to claim 6, wherein the subject may be further tested one or more times at intervals of time until no FLC clone is detected by MS.

8. A method according to claim 7, wherein if no clone is detected then a sample of bone marrow is tested for a clone.

9. A method according to claim 1, wherein MS is liquid chromatography MS or MALDI-TOF.

10. A method according to claim 1, wherein the sample is a sample of blood, serum, plasma, cerebrospinal fluid, or urine.

11. A method according to claim 1, wherein the monoclonal gammopathy is selected from multiple myeloma, LA amyloidosis plasmacytoma, Waldenström's macroglobulinaemia, B-cell non-Hodgkin lymphoma, and B-cell chronic lymphocytic leukaemia.

12. A method according to claim 1, wherein the subject is monitored by one or more additional techniques to monitor immunoglobulins in the sample prior to detecting a FLC clone by MS.

13. A method according to claim 12, wherein the presence of free light chain in the subject is monitored by nephelometry, turbidimetry or ELISA and FLC-MS screening is used once the presence of free light chains is observed to return to a predetermined normal level in the subject.

14. A method according to claim 12, wherein the immunoglobulins in the subject are detected by serum plasma electrophoresis (SPE) or immunofixation electrophoresis (IFE) and the FLC-MS screening is carried out if the (SPE) or IFE is identified not to be normal.
